SEQUENCE LISTING (2 sequences)

SEQ ID NO: 1 - protein, 501 aa, Homo sapiens
MVRKPVVSTISKGGYLQGNVNGRLPSLGNKEPPGQEKVQLKRKVTLLRGVSIIIGTIIGAGIFISPKGVLQNTGSVGMSLTIWTVCGVLSLFGALSYAELGTTIKKSGGHYTYILEVFGPLPAFVRVWVELLIIRPAATAVISLAFGRYILEPFFIQCEIPELAIKLITAVGITVVMVLNSMSVSWSARIQIFLTFCKLTAILIIIVPGVMQLIKGQTQNFKDAFSGRDSSITRLPLAFYYGMYAYAGWFYLNFVTEEVENPEKTIPLAICISMAIVTIGYVLTNVAYFTTINAEELLLSNAVAVTFSERLLGNFSLAVPIFVALSCFGSMNGGVFAVSRLFYVASREGHLPEILSMIHVRKHTPLPAVIVLHPLTMIMLFSGDLDSLLNFLSFARWLFIGLAVAGLIYLRYKCPDMHRPFKVPLFIPALFSFTCLFMVALSLYSDPFSTGIGFVITLTGVPAYYLFIIWDKKPRWFRIMSEKITRTLQIILEVVPEEDKL

SEQ ID NO: 2 - protein, 502 aa, Mus sp.
MVRKPVVATISKGGYLQGNMSGRLPSMGDQEPPGQEKVVLKKKITLLRGVSIIIGTVIGSGIFISPKGILQNTGSVGMSLVFWSACGVLSLFGALSYAELGTSIKKSGGHYTYILEVFGPLLAFVRVWVELLVIRPGATAVISLAFGRYILEPFFIQCEIPELAIKLVTAVGITVVMVLNSTSVSWSARIQIFLTFCKLTAILIIIVPGVIQLIKGQTHHFKDAFSGRDTSLMGLPLAFYYGMYAYAGWFYLNFITEEVDNPEKTIPLAICISMAIITVGYVLTNVAYFTTISAEELLQSSAVAVTFSERLLGKFSLAVPIFVALSCFGSMNGGVFAVSRLFYVASREGHLPEILSMIHVHKHTPLPAVIVLHPLTMVMLFSGDLYSLLNFLSFARWLFMGLAVAGLIYLRYKRPDMHRPFKVPLFIPALFSFTCLFMVVLSLYSDPFSTGVGFLITLTGVPAYYLFIVWDKKPKWFRRLSDRITRTLQIILEVVPEDSKEL

(19) United States
(12) Patent Application Publication
(10) Pub. No.: US 2022/0088040 A1
(43) Pub. Date: Mar. 24, 2022

(54) Use of System XC-Inhibitor for Treating Statin-Induced Myalgia

(71) Applicant: Exerkine Corporation, Hamilton (CA)

(72) Inventors: Mark Tarnopolsky, Hamilton (CA); Thomas Hawke, Hamilton (CA)

(21) Appl. No.: 16/755,631

(22) PCT Filed: Oct. 12, 2018
(86) PCT No.: PCT/CA2018/051285

§ 371 Date: Apr. 13, 2020

Related U.S. Application Data
(60) Provisional application No. 62/572,029, filed on Oct. 13, 2017.

(51) Int. Cl. A61K 31/655; A61K 31/355; A61K 31/122; A61K 31/145; A61K 31/51; A61K 31/525; A61K 31/385; A61K 31/205; A61K 36/21; A61P 29/00

(52) U.S. Cl. CPC A61K 31/655; A61K 31/355; A61K 31/122; A61K 31/145; A61P 29/00; A61K 31/525; A61K 31/385; A61K 31/205; A61K 36/21; A61K 31/51

(57) ABSTRACT

A method of reducing glutamate efflux from skeletal muscle by inhibiting system Xc- activity is provided. The method is useful for the treatment of statin-induced myalgia. Pharmaceutical compositions useful to treat statin-induced myalgia are also provided, as well as a kit.

### FIELD OF THE INVENTION

The present invention generally relates to statin-induced myalgia, and more particularly relates to a method of reducing glutamate efflux from muscle cells for the treatment of statin-induced myalgia.

### BACKGROUND OF THE INVENTION

Statins are a class of cholesterol-lowering drugs commonly used for the treatment of hypercholesterolemia, which act by competitively inhibiting 3-hydroxy-3-methylglutaryl coenzyme A reductase (HMGCR). As HMGCR is a rate-determining enzyme in the biosynthesis of the cholesterol precursor molecule, mevalonate, statins function to reduce cholesterol synthesis. Statins have also been found to lower circulating cholesterol levels by increasing expression of the hepatic low density lipoprotein (LDL) cholesterol receptor, which consequently increases liver uptake of LDL cholesterol. Elevated cholesterol is widely established as a primary factor for the development of cardiovascular disease such as coronary artery disease and cardiac events. Due to their highly potent effects, statins have become the standard of care for treating elevated cholesterol and are now one of the most commonly prescribed drugs worldwide.

Although well tolerated, there are side effects associated with statin therapy, for example, statin-induced myopathy. Myopathy is a term used to refer to genetic or acquired disorders of skeletal muscle. Symptoms of myopathy can include; muscle weakness, exercise-induced fatigue, and rhabdomyolysis or myalgia (muscle pain). Statin-induced myopathies encompass a wide spectrum of muscle-related symptoms such as myalgia, myositis and rhabdomyolysis. Of these, statin-induced myalgia or muscle pain is the most commonly reported side effect of statin therapy; although the mechanism(s) is/are not well understood. Observational studies have reported between 1 and 29% of individuals taking statins complain of myalgia and it is not clear why statins cause myalgia.

One metabolite of statin that is capable of causing pain in skeletal muscle is the amino acid, glutamate. This pain response results from the binding of glutamate to peripheral pain receptors (or nociceptors) in skeletal muscle, but it is unknown if glutamate levels are related to statin-induced myalgia. Numerous risk factors have been identified as placing individuals at a higher risk for statin-induced myalgia including: high statin dosages, reduced muscle mass, advanced age, excessive exercise, excessive alcohol consumption, liver disease, renal failure and hypothyroidism. There is presently no cure or effective treatment for statin-induced myalgia. The current method of treatment relies on reducing the statin dose, altering the frequency of statin administration or using alternative cholesterol lowering drugs such as fibric acid derivatives, bile acid binding resins, or ezetimibe. The development of statin-induced myalgia can pose a significant burden on individuals by reducing quality of life, mobility, muscle strength and physical activity. Statin-induced myalgia also commonly results in discontinuation of the statin therapy given that alterations in statin dose, type, frequency or combinations rarely alleviate the myalgia symptoms and the less effective alternative drugs remain the only treatment option. Consequently, statin intolerance represents a serious concern and obstacle for healthcare providers in the effective management of hypercholesterolemia and cardiovascular disease as there is no similarly effective treatment for elevated cholesterol levels.

It would be desirable, thus, to provide an effective method for the treatment of statin-induced myalgia.

### SUMMARY OF THE INVENTION

It has now been found that the reduction of glutamate efflux from skeletal muscle is useful to treat statin-induced myalgia.

Thus, in a first embodiment of the present invention, a method of reducing glutamate efflux from cells is provided comprising administering to the cells a system Xc-inhibitor.

In another embodiment of the present invention, a method of treating statin-induced myalgia in a mammal is provided, comprising administering to the mammal a therapeutically effective amount of a composition which inhibits system Xc- activity.

In another embodiment of the invention, a method of treating statin-induced myalgia in a mammal is provided, comprising administering to the mammal a therapeutically effective amount of a system Xc- inhibitor.

In another embodiment of the invention, a pharmaceutical composition for inhibiting system Xc- activity in a mammal is provided comprising a system Xc- inhibitor cocktail comprising a combination of two or more of the following inhibitors: sulfasalazine, vitamin E, coenzyme Q10 and cysteanine.

In another embodiment of the invention, a kit is provided comprising a pharmaceutical composition for inhibiting system Xc- activity and one or more of the following: a statin, a compound effective to treat mitochondrial dysfunction or a compound effective to treat muscle pain.

In another embodiment of the invention, a method of treating fibromyalgia in a mammal is provided, comprising administering to the mammal a therapeutically effective amount of one or more system Xc- inhibitors.

These and other aspects of the present invention will become apparent in the detailed description that follows, by reference to the following figures.

### BRIEF DESCRIPTION OF THE FIGURES

FIG. 1 graphically illustrates xCT protein content in C2C12 myotubes treated with either atorvastatin or vehicle. n=5-8 wells per group over 4 rounds of experimentation. * Indicates a significant (P<0.05) difference from the indicated group(s).

FIG. 2 graphically illustrates glutamate efflux from C2C12 myotubes treated with either atorvastatin, vehicle, sulfasalazine or an atorvastatin-sulfasalazine co-treatment. n=5-8 wells per group over 4 rounds of experimentation. * Indicates a significant (P<0.05) difference from the indicated group(s).

FIG. 3 graphically illustrates glutamate efflux from A) primary human myoblasts treated with either atorvastatin, vehicle or an atorvastatin-sulfasalazine co-treatment and B) primary human fibroblasts treated with either atorvastatin, vehicle or an atorvastatin-sulfasalazine co-treatment.

FIG. 4 graphically illustrates glutamate efflux from C2C12 myotubes treated with the statin, atorvastatin (5μM), or with the statin simultaneously with each of the following: A) sulfasalazine, B) cysteamine bitartrate, C) vitamin E, D) coenzyme Q10, E) vitamin E and coenzyme Q10, and F) N-acetylcysteine (NAC), as compared to vehicle. n=23 wells per group over 5 rounds of experimentation for statin group. n=3-9 for all other groups over 2 rounds of experimentation. Values for statin alone treatments are each derived from the same pooled results obtained over 5 rounds of experimentation. * Indicates a significant (P<0.05) difference from the indicated group(s).

FIG. 5 graphically illustrates glutamate efflux from C2C12 myotubes treated with the statin, atorvastatin (7.5 μM), or with the statin simultaneously with each of the following: A) sulfasalazine, B) cysteamine bitartrate, C) vitamin E, D) coenzyme Q10, E) vitamin E and coenzyme Q10, and F) N-acetylcysteine (NAC). n=27 wells per group over 5 rounds of experimentation for statin group. n=3-7 for all other groups over 2 rounds of experimentation. Relative values for statin alone treatments are each derived from the same pooled results obtained over 5 rounds of experimentation. * Indicates a significant (P<0.05) difference from the indicated group(s).

FIG. 6 graphically illustrates glutamate efflux from the extramyocellular fluid of muscle from rats treated with statins or various system Xc- inhibitors.

FIG. 7 illustrates the amino acid sequence of human (A) and mouse (B) system Xc-.

### DETAILED DESCRIPTION OF THE INVENTION

A method of reducing glutamate efflux from skeletal muscle is provided for the treatment of statin-induced myalgia. In one embodiment, the method comprises reducing glutamate efflux from cells (e.g. muscle cells such as skeletal cells) by administering to the cells a system Xc- inhibitor.

The term “glutamate efflux” is used herein to describe the outward movement of glutamate from the intramyocellular space to the extramyocellular space.

The term “reducing” as it is used with respect to glutamate efflux, refers at least to a lowering of the total net amount of glutamate being transferred into the extramyocellular space, for example, by at least about 10% of the glutamate efflux occurring following statin administration, and preferably a lowering of glutamate efflux by about 25% or more, e.g. by 40%, 50%, 60%, 70%, 80% or greater, e.g. a lowering of glutamate efflux to the baseline level present prior to statin administration. The term “about” as used herein refers to a variation from the indicated amount of 10% or less, preferably 5% or less.

The term “statin-induced myalgia” is used herein to refer to the sensation of pain experienced by a mammal that can reasonably be attributed to administration of a statin. Statin-induced myalgia can occur in the presence or absence of comorbidities or other common statin-induced side effects such as elevated creatine kinase levels, myositis or rhabdomyolysis.

The term “statin” is used herein to refer to any pharmaceutical compound which inhibits the activity of 3-hydroxy-3-methylglutaryl coenzyme A reductase (HMGCR) or otherwise prevents or reduces the formation of mevalonate by HMGCR. Examples of statins include, but are not limited to, the following: atorvastatin, lovastatin, simvastatin, mevinolin, compactin, cerivastatin, synvinolin, velostatin, fluvastatin, verivastatin, pitaviastatin, pravastatin, rivastatin, rosuvastatin and mevastatin. As the field of high blood lipid treatment is constantly evolving and new or modified statin drugs are being developed on a continual basis, the term “statin” as used herein is intended to include those statins which have yet to be developed.

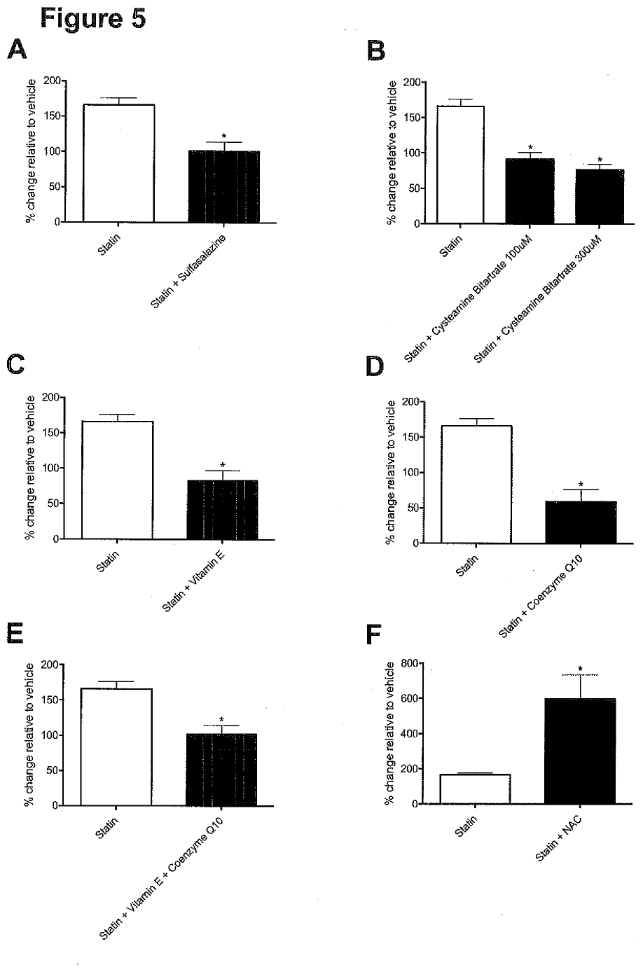

The term “system Xc-” is used herein to encompass mammalian system Xc- (e.g. the wildtype isoform), including human (see FIG. 5A) and functionally equivalent forms thereof, including isoforms, variants and non-human forms (see FIG. 5B) of system Xc-. System Xc- is encoded by the gene, SLC7A11, the human sequence of which is known and available at the National Centre of Biotechnology Information (NCBI), reference NC_000004.12, and the corresponding mouse sequence is NCBI reference, NC_000069.6. The term “functionally equivalent forms” is used herein to refer to a modified form of a functional wildtype system Xc-which substantially retains the activity. A functionally equivalent form may not necessarily exhibit equivalent activity to the wildtype compound, but retains a substantial amount activity, e.g. about 50% of the activity of the wildtype compound. The system Xc- protein is also commonly referred to by several other names including, but not limited to, the following: amino acid transport system xc-, cystine/glutamate transporter, solute carrier family 7 member 11 and cystine-glutamate antiporter.

System Xc- is an antiporter transport protein which exchanges cystine and glutamate across the myocellular membrane in opposing directions at a ratio of 1:1. The directionality of amino acid exchange by the system Xc- protein is believed to be governed primarily by the relative concentration gradients of cystine and glutamate on each side of the myocellular membrane. System Xc- is a heterodimeric protein consisting of an xCT protein subunit and 4F2 cell-surface antigen heavy chain (4F2hc) protein subunit.

The term “activity” as it is used herein with respect to system Xc-, refers to the total net export of glutamate from the intramyocellular space to the extramyocellular space by system Xc.

Glutamate efflux from cells is reduced in accordance with an aspect of the invention by administering to the cells a system Xc- inhibitor. The term “system Xc- inhibitor” is used herein to refer to any agent or composition that inhibits or at least reduces system Xc- activity, and the resulting glutamate efflux, by at least about 10% of the system Xc- activity occurring following statin administration, and preferably a reduction of system Xc- activity by about 25% or more, e.g. by 40%, 50%, 60%, 70%, 80% or greater, e.g. a lowering of system Xc- activity to the baseline level present prior to statin administration.

System Xc- inhibitors for use in the present method include small molecule inhibitors such as, but not limited to, sulfasalazine, cysteamine, methylene blue, coenzyme Q10, vitamin E, erastin, sorafenib, regorafenib, L-lactate, L-cystine, L-glutamate, D-serine-O-sulphate, L-alpha-aminoadipate, L-alpha-aminopimelate, L-homocysteate, S-sulpho-L-cysteine, L-serine-O-sulphate, L-homocysteine sulphinate, L-beta-N-oxalyl-L-alpha,beta-diaminopropionate (beta-L-ODAP), L-alanosine, quisqualate, ibotenate, (RS)-4-Br-homoibotenate, S-2-naphthyl-ethyl-amino-3-carboxy-5-methyl isoxazole propionic acid (NACPA), bis-trifluoromethylphenyl-isoxazole-4-hydrazone (TFMIH), 5-naphthylethyl isoxazole-4-(2,4-dinitrophenol)hydrazone-dinitrophenol (NEIH), (S)-4-carboxyphenyglycine (4-S-CPG), sulphonic acid phenylglycine (4-S-SPG), (R,S)-4-[4′-carboxyphenyl]-phenyiglyeine (CPPG), (2E)-N-[(5-bromo-2-methoxyphenyl)sulfonyl]-3-[2-(2-naphthalenylmethyl)phenyl]-2-propenamide (L-798106), 4-[(1E)-2-(5,6,7,8-tetrahydro-5,5,8,8-tetramethyl-2-naphthalenyl)-1-propen-1-yl]-benzoic acid (TTNPB), candesartan cilextil, SKF 38393, capsazepine, mesalamine, osalazine, balsalazide, and combinations thereof. Other system Xc- inhibitors include beet root extract, alpha lipoic acid, creatine, green tea extract, black tea extract, green coffee bean extract, conjugated linoleic acid and forskolin.

Pharmaceutically acceptable salts of such small molecule inhibitors are also encompassed, including acid and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids, tartaric acid, and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. For example, acceptable salts of cysteamine include, but are not limited to: cysteamine hydrochloride, phosphocysteamine, and cysteamine bitartrate.

Functionally equivalent isomers of system Xc- inhibitors for use in the present method are also encompassed including stereoisomers thereof such as enantiomers and diastereomers. For example, Vitamin E encompasses isomers such as alpha-tocopherol, beta-tocopherol, gamma-tocopherol, delta-tocopherol, alpha-tocotrienol, beta-tocotrienol, gamma-tocotrienol, and delta-tocotrienol. Preferably, the form of vitamin E used is alpha-tocopherol, which may comprise any of the biologically functional stereoisomers of alpha-tocopherol such as the naturally occurring RRR-configuration or the synthetically produced 2R-stereoisomer forms (RSR-, RRS-, and RSS-).

In addition, functionally equivalent redox states of system Xc- inhibitors are encompassed for use in the present method. For example, coenzyme Q10, also known as ubiquinone, ubidecarenone, coenzyme Q, CoQ10, CoQ, or Q10, may assume any one of three redox states, namely, fully oxidized (ubiquinone), semi-oxidized (semiquinone or ubisemiquinone), and fully reduced (ubiquinol), or oxidized mitochondrially targeted forms of this enzyme (e.g. mitoquinone mesylate (MitoQ₁₀)). As would be appreciated by one of skill in the art, coenzyme Q10 can be formulated in numerous ways to improve the bioavailability or effectiveness of coenzyme Q10 treatment. Examples of such formulations, which are not intended to be limiting, include the following: colloidal-based, solid dispersion-based, oily dispersion-based, micelle-based, nanoliposome-based, nanostructured lipid carrier-based, nanocrystal-based, nanoparticle-based, self-nanoemulsifiable-based, ascorbic acid with chelation-based and cyclodextrin complexation-based.

To reduce glutamate efflux in the treatment of statin-induced myalgia, a therapeutically effective amount of a system Xc- inhibitor is administered to a mammal. As used herein, the term “mammal” is meant to encompass, without limitation, humans, domestic animals such as dogs, cats, horses, cattle, swine, sheep, goats and the like, as well as non-domesticated animals such as, but not limited to, mice, rats and rabbits. The terms “treat”, “treating” or “treatment” are used herein to refer to methods that favorably alter a pathological condition such as statin-induced myalgia, including those that moderate, reverse, reduce the severity of, or protect against, the progression of statin-induced myalgia. The term “therapeutically effective amount” is an amount of the system Xc- inhibitor required to reduce glutamate efflux by at least about 10% or greater of the statin-induced glutamate efflux, for example, in muscle, while not exceeding an amount which may cause significant adverse effects, to result in a reduction of statin-induced myalgia by an amount of at least 10%, but preferably by an amount of at least 20%, 30%, 40%, 50%, 60%, 70%, 80%, 90% or greater. Dosages of system Xc- inhibitors that are therapeutically effective will vary on many factors including the severity of myalgia experienced as well as the particular individual being treated. The dosages of system Xc- inhibitors that are therapeutically effective also depend on the type of system Xc- inhibitor in use. Appropriate dosages of sulfasalazine for use include dosages within the range of about 250 mg to about 5,000 mg, for example, 1,000 mg to about 2,000 mg. Appropriate dosages of Vitamin E for use include dosages within the range of about 25 IU to about 2,500 IU, for example, 400 IU to about 800 IU. Appropriate dosages of cysteamine for use include dosages within the range of about 150 mg to about 6,000 mg, for example, 300 mg to about 2,400 mg. Appropriate dosages of coenzyme Q10 for use include dosages within the range of about 25 mg to about 1,000 mg, for example, 100 mg to about 400 mg. The system Xc- inhibitor may be formulated in a dose which would be appropriate for administration at a rate of one or more doses per day. In one embodiment, the system Xc- inhibitor may be formulated in a sustained release system wherein the tissue or blood levels of the active agent are prolonged. The system Xc- inhibitor may also be formulated in a controlled release system wherein the release of the active agent is controlled spatially, temporally or in a combination thereof.

In one embodiment, the present method comprises administration of a composition of at least two system Xc- inhibitors selected from vitamin E, coenzyme Q10, beet root extract, alpha lipoic acid and creatine. In a further embodiment, the method comprises administration of a composition of at least two system Xc- inhibitors selected from vitamin E, coenzyme Q10, beet root extract, alpha lipoic acid, creatine, green tea extract, black tea extract, green coffee bean extract, conjugated linoleic acid and forskolin.

System Xc- inhibitor compositions may comprise about 0.1-50% vitamin E of the dry weight of the system Xc- inhibitor composition, such as about 1-20% vitamin E, about 2-5% vitamin E of the dry weight of the composition, or about 10 mg-1 g of vitamin E and preferably, about 50-200 mg vitamin E.

System Xc- inhibitor compositions may comprise about 0.1-50% coenzyme Q10 of the dry weight of the system Xc- inhibitor composition, such as about 1-20% coenzyme Q10, 2-5% coenzyme Q10 of the dry weight of the composition, or about 10 mg-1 g of coenzyme Q10 and preferably, about 50-200 mg coenzyme Q10.

The beetroot extract for use in the present composition may be selected from any suitable beetroot source including red beets such as Detroit Dark Red, Red Ace, Early Wonder Tall Top, Bull's Blood, Forono, Ruby Queen, Chioggia, Cylindra or Gladiator, yellow or gold beets such as Yellow Detroit, Golden, Touchstone Gold or Boldor or white beets such as Avalanche, Baby White, Blankoma or Sugar. Preferably, the beetroot extract is substantially derived from the taproot portion of the beetroot. In one embodiment, the beetroot extract contains at least 1.5% nitrates by dry weight. In another embodiment, the beetroot extract comprises about 0.1-50% of the dry weight of a system Xc- inhibitor composition for use in the present method, such as about 1-20%, or about 5-10% of the dry weight of the composition. In a further embodiment, the system Xc- inhibitor composition comprises about 10 mg-50 g of beetroot extract and preferably, about 100-1000 mg.

Alpha lipoic acid suitable for use in the present composition may include, without limitation, alpha lipoic acid or its reduced form, dihydrolipoic acid, with R- and S-enantiomers either present individually, in racemic form or in any other mixture thereof. The R-enantiomer is produced naturally or synthetically, while the S-enantiomer is only produced synthetically and does not occur naturally. Additionally, any pharmaceutically acceptable salts or derivatives thereof are suitable for use in the present method. Preferably, the alpha lipoic acid is in racemic form. In one embodiment, the alpha lipoic acid comprises about 0.1-50% of the dry weight of a system Xc- inhibitor composition for use in the present method, such as about 1-20%, or about 2-5% of the dry weight of the composition. In another embodiment, the system Xc- inhibitor composition comprises about 10 mg-3 g of alpha lipoic acid and preferably, about 50 mg-500 mg.

Creatine for use in the method may be in any suitable form, such as creatine monohydrate, creatine anhydrous, creatine citrate, creatine ethyl ester, creatine nitrate, creatine magnesium chelate, creatine hydrochloride, creatine malate, creatine pyruvate, creatine phosphate, creatine citrate malate, creatine tartrate, creatine HMB (β-hydroxy β-methylbutyrate), effervescent creatine, creatine titrate, buffered creatine, micronized creatine and any combination thereof. Preferably, the creatine is creatine monohydrate. In one embodiment, creatine comprises about 1%-80% of the dry weight of a system Xc- inhibitor composition for use in the present method, such as about 20-70%, or about 30-50% of the dry weight of the composition. In another embodiment, the system Xc- inhibitor composition comprises about 0.1-10 g of creatine and preferably, about 1-5 g.

The green tea extract for use in the present method is selected from any suitable green tea leaf or green tea source such as Sencha, Fukamushi Sencha, Gyokuro, Kabusecha, Matcha, Tencha, Genmaicha, Matcha, Shincha, Hojicha, Ichibanchagreen, Nibancha and Sanbancha tea, which are derived from the Camellia sinensis leaf. Green tea is abundant in polyphenols such as catechins. Examples of such catechins include catechin, catechin gallate, epicatechin, gallocatechin, epigallocatechin, and epicatechin gallate. Preferably, the green tea extract contains 10% or more of catechins by dry weight. Green tea extract for use in the present method may be either caffeinated or substantially decaffeinated, for example, having less than 1% of caffeine by dry weight. Preferably, the green tea extract contains 30% caffeine by dry weight and 20% catechins by dry weight. In one embodiment, the green tea extract comprises about 0.1-50% of the dry weight of a system Xc- inhibitor composition for use in the present method, such as about 1-20%, or about 2-5% of the dry weight of the composition. In another embodiment, the system Xc- inhibitor composition comprises about 10 mg-5 g of green tea extract and preferably, about 50-500 mg.

The black tea extract may be selected from any suitable black tea leaf or black tea source including unblended black tea sources such as Congou, Assam, Darjeeling, Nilgiri or Ceylon or blended black teas such as Earl Grey, English Breakfast tea, English afternoon tea, Irish breakfast tea or Masala chai, which are derived from the Camilla sinensis leaf. Black tea is abundant in polyphenols such as theaflavins, thearubigins and catechins. Examples of theaflavins include theaflavin, theaflavin-3-gallate, theaflavin-3′-gallate and theaflavin-3,3′-gallate. Preferably, the black tea extract contains 10% or more of polyphenols by dry weight. Black tea extract for use in the present method may be either caffeinated or substantially decaffeinated, for example, having less than 1% of caffeine by dry weight. Preferably, the black tea extract contains at least 30% polyphenols by dry weight. In one embodiment, the black tea extract comprises about 0.1-50% of the dry weight of a system Xc- inhibitor composition for use in the present method, such as about 1-20%, or about 2-5% of the dry weight of the composition. In another embodiment, the system Xc- inhibitor composition comprises about 10 mg-5 g of black tea extract and preferably, about 50-500 mg.

The green coffee bean extract for use is selected from any suitable green coffee bean source such as Coffea Arabica or Coffea canephora. Green coffee beans contain several types of chlorogenic acids, such as 3-caffeoylquinic acid, 4-caffeoylquinic acid and 5-caffeoylquinic acid. Preferably, the green coffee bean extract contains 30% or more of chlorogenic acids by dry weight. Green coffee bean extract for use in the present method may be either caffeinated or substantially decaffeinated, for example, having less than 1% of caffeine by dry weight. Preferably, the green coffee bean extract contains at least 50% chlorogenic acids and less than 4% caffeine by dry weight. In one embodiment, the green coffee bean extract comprises about 0.1-50% of the dry weight of a system Xc- inhibitor composition for use in the present method, such as about 1-20%, or about 2-5% of the dry weight of the composition. In another embodiment, the system Xc- inhibitor composition comprises about 10 mg-5 g of green coffee bean extract and preferably, about 50-500 mg.

The conjugated linoleic acid may be selected from any suitable source such as safflower oil, sunflower oil or grass-fed beef sources. As used herein, the term “conjugated linoleic acid” refers to any of the at least 28 known geometric or positional isomers of linoleic acid, wherein two of the double bonds of the molecule are conjugated such as in the cis-9:trans-11 or trans-10:cis-12 form. A system Xc- inhibitor composition for use in the present methods may include a single isomer, a mixture of isomers, natural isomers, synthetic isomers, or a pharmaceutically acceptable salt, ester, monoglyceride, diglyceride, triglyceride, metabolic precursor thereof, or any combinations thereof. Preferably, the conjugated linoleic acid contains about a 50:50 mixture of its cis-9:trans-11, and trans-10:cis-12 isomers. In one embodiment, the conjugated linoleic acid source comprises about 1%-80% of the dry weight of the system Xc- inhibitor composition composition, such as about 20-70%, or about 30-50% of of the dry weight of the composition. In another embodiment, the system Xc- inhibitor composition comprises about 10 mg-10 g of conjugated linoleic acid and preferably, about 500 mg-3 g.

The forskolin for use in the present method is selected from any suitable source. Forskolin may be extractred from the Coleus forskohli plant, or synthetically produced. Preferably, the forskolin extract for use is derived from the Coleus forskohli plant and is standardized to contain 40% forskolin. In one embodiment, forskolin comprises about 0.05-10% of the dry weight of a system Xc- inhibitor composition for use in the present method, such as about 0.1-5%, or about 0.2-1% of the dry weight of the composition. In another embodiment, the system Xc- inhibitor composition comprises about 1 mg-200 mg of forskolin and preferably, about 15 mg-50 mg.

In one embodiment, a system Xc- inhibitor composition for use in the present method comprises 50-200 mg of vitamin E, 50-200 mg of coenzyme Q10, 100-1000 mg of beetroot extract, 50 mg-500 mg alpha lipoic acid and 1-5 g of creatine.

In another embodiment, the system Xc- inhibitor composition comprises 50-200 mg of vitamin E, 50-200 mg of coenzyme Q10, 100-1000 mg of beetroot extract, 50 mg-500 mg alpha lipoic acid, 1-5 g of creatine, 50-500 mg of green tea extract, 50-500 mg of black tea extract, 50-500 mg of green coffee bean extract, 500 mg-3 g of conjugated linoleic acid and 15 mg-50 mg of forskolin.

In another embodiment, the system Xc- inhibitor composition comprises 50-200 mg of vitamin E, 50-200 mg of coenzyme Q10, 100-1000 mg of beetroot extract, 50 mg-500 mg alpha lipoic acid, 50-500 mg of green tea extract, 50-500 mg of green coffee bean extract and 15 mg-50 mg of forskolin.

In yet another embodiment, the system Xc- inhibitor composition comprises 50-200 mg of vitamin E, 50-200 mg of coenzyme Q10, 100-1000 mg of beetroot extract, 50 mg-500 mg alpha lipoic acid, 1-5 g of creatine, 50-500 mg of green tea extract, 50-500 mg of green coffee bean extract and 15 mg-50 mg of forskolin.

In accordance with the present invention, the system Xc- inhibitors may also be compounds that inhibit the expression or in vivo stability of system Xc- mRNA. For example, nucleic acid-based inhibitors may be used to inhibit system Xc-, such as anti-sense inhibitors and RNA interference inhibitors, e.g. siRNA, shRNA and the like. Knowledge of the system Xc-encoding nucleic acid sequence may be used to prepare antisense oligonucleotides effective to bind to system Xc- nucleic acid and inhibit the expression thereof. The term “antisense oligonucleotide” as used herein means a nucleotide sequence that is complementary to at least a portion of a target system Xc- nucleic acid sequence. The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells) as well as the antisense binding region. In addition, two or more antisense oligonucleotides may be linked to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydrodyl guanine and other 8-substituted guanines, other aza and deaza uracils, thymidines, cytosines, adenines, or guanines, 5-tri-fluoromethyl uracil and 5-trifluoro cytosine. Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates and phosphorodithioates. In addition, the antisense oligonucleotides may contain a combination of linkages, for example, phosphorothioate bonds may link only the four to six 3′-terminal bases, may link all the nucleotides or may link only 1 pair of bases.

The antisense oligonucleotides of the invention may also comprise nucleotide analogs that may be better suited as therapeutic agent. An example of an oligonucleotide analogue is a peptide nucleic acid (PNA) in which the deoxribose (or ribose) phosphate backbone in the DNA (or RNA), is replaced with a polyamide backbone which is similar to that found in peptides. PNA analogues have been shown to be resistant to degradation by enzymes and to have extended lives in vivo and in vitro. PNAs also form stronger bonds with a complementary DNA sequence due to the lack of charge repulsion between the PNA strand and the DNA strand. Other oligonucleotide analogues may contain nucleotides having polymer backbones, cyclic backbones, or acyclic backbones. For example, the nucleotides may have morpholino backbone structures (U.S. Pat. No. 5,034,506). Oligonucleotide analogues may also contain groups such as reporter groups, protective groups and groups for improving the pharmacokinetic properties of the oligonucleotide. Antisense oligonucleotides may also incorporate sugar mimetics as will be appreciated by one of skill in the art.

Antisense nucleic acid molecules may be constructed using well-established chemical and enzymatic ligation reactions. The antisense nucleic acid molecules of the invention, or fragments thereof, may be chemically synthesized using naturally occurring nucleotides or variously modified nucleotides designed to increase the biological stability of the molecules or to increase the physical stability of the duplex formed with mRNA or the native gene, e.g. phosphorothioate derivatives and acridine substituted nucleotides. The antisense sequences may also be produced biologically. In this case, an antisense encoding nucleic acid is incorporated within an expression vector that is then introduced into cells in the form of a recombinant plasmid, phagemid or attenuated virus in which antisense sequences are produced under the control of a high efficiency regulatory region, the activity of which may be determined by the cell type into which the vector is introduced.

In another embodiment, RNA silencing technology can be applied to inhibit system Xc- expression. Application ofnucleic acid fragments such as siRNA and shRNA fragments that correspond with and selectively target regions in a system Xc- transcript may be used to block system Xc-expression. Such blocking occurs when the siRNA or shRNA fragments bind to the transcript thereby preventing translation thereof to yield functional system Xc-. SiRNA, small interfering RNA molecules, or shRNA, small hairpin RNA molecules, corresponding to system Xc- mRNA are made using well-established methods of nucleic acid syntheses as outlined above with respect to antisense oligonucleotides. The effectiveness of selected siRNA and shRNA to block system Xc- expression can be confirmed using a system Xc-expressing cell line. Briefly, selected siRNA/shRNA may be incubated with a system Xc-expressing cell line under appropriate growth conditions. Following a sufficient reaction time, i.e. for the siRNA or shRNA to bind with system Xc- mRNA to result in decreased system Xc- expression, the reaction mixture is tested to determine if such a decrease has occurred. Suitable siRNA/shRNA will prevent processing of the system Xc- transcript to yield functional system Xc- protein. This can be detected by assaying for system Xc- activity in a cell-based assay, for example, to identify expression of a reporter gene that is regulated by system Xc- binding.

It will be appreciated by one of skill in the art that siRNA/shRNA fragments useful in the present method may be derived from specific regions of system Xc--encoding nucleic acid which may provide more effective inhibition of gene expression, for example, the 3′ end of the transcript, including the 3′ untranslated portion. In addition, as one of skill in the art will appreciate, useful siRNA fragments may not correspond exactly with a region of the system Xc-target gene, but may incorporate sequence modifications, for example, addition, deletion or substitution of one or more of the nucleotide bases therein, provided that the modified siRNA retains its ability to bind to the target gene. Selected siRNA fragments may additionally be modified in order to yield fragments that are more desirable for use. For example, siRNA fragments may be modified to attain increased stability in a manner similar to that described for antisense oligonucleotides.

System Xc- may also be inhibited using compounds that post-translationally modify system Xc- proteins to yield non-functional system Xc-. Examples of common types of post-translational modifications that result in non-functional system Xc- include but are not limited to: phosphorylation, acetylation, N-linked glycosylation, amidation, hydroxylation, methylation, O-linked glycosylation, ubiquitylation, pyrrolidone carboxylic acid modification and sulfation.

As would be appreciated by a person of skill in the art, immunological polypeptides, proteins or functionally equivalent fragments thereof may be used as inhibitors of system Xc-activity. Such polypeptides, proteins or functionally equivalent fragments thereof generally inhibit system Xc- proteins by binding to functional domains of a system Xc- protein. Examples of suitable immunological polypeptides include, but are not limited to the following: dominant negative system Xc- fragments, polypeptide binding functional domains such as at the lipophilic binding domains, monoclonal antibodies, chimeric antibodies, humanized antibodies, polyclonal antibodies, functionally equivalent derivatives of said antibodies or antigen-binding fragments of said antibodies. Antibodies may be prepared using well-established hybridoma technology. For example, antibodies may be made by injecting a host animal, e.g. a mouse or rabbit, with a system Xc-antigenic peptide, and then isolating antibodies generated by the animal from a biological sample taken therefrom. Alternatively, antibodies may be conmmercially obtained, e.g. from Abeam, Novus Biologicals, Invitrogen, etc.

System Xc- inhibitors may be administered either alone or in combination with at least one pharmaceutically acceptable adjuvant, for use in treatments in accordance with embodiments of the invention. The expression “pharmaceutically acceptable” means acceptable for use in the pharmaceutical and veterinary arts, i.e. not being unacceptably toxic or otherwise unsuitable. Examples of pharmaceutically acceptable adjuvants include diluents, excipients and the like. Reference may be made to “Remington's: The Science and Practice of Pharmacy”, 21st Ed., Lippincott Williams & Wilkins, 2005, for guidance on drug formulations generally. The selection of adjuvant depends on the intended mode of administration of the composition. In one embodiment of the invention, the compounds are formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. In another embodiment, the present composition is formulated for oral administration, The term “oral” or “orally” as used herein is intended to include any method in which the system Xc- inhibitor is introduced into the digestive tract including the stomach and small intestine. Examples of oral administration may include administration via mouth, directly into the stomach using a feeding tube, through the nose to the stomach via a feeding tube and through the nose to the small intestine via a feeding tube. Compositions for oral administration via tablet, capsule, powder, suspension or solution are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerin, sorbital and mannitol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Creams, lotions and ointments may be prepared for topical application using an appropriate base such as a triglyceride base. Such creams, lotions and ointments may also contain a surface active agent. Aerosol formulations may also be prepared in which suitable propellant adjuvants are used. Other adjuvants may also be added to the composition regardless of how it is to be administered for example, anti-microbial agents may be added to the composition to prevent microbial growth over prolonged storage periods. The composition may include a coating or may be encased in a protective material to prevent undesirable degradation thereof by enzymes, acids or by other conditions that may affect the therapeutic activity thereof.

To treat statin-induced myalgia, a system Xc- inhibitor may be administered in conjunction with one or more statins. The term “in conjunction with” as used herein refers to any of the various means and temporal arrangments by which two or more agents may be administered. The system Xc- inhibitor and statin(s) may be formulated together as a single composition, or administered separately in distinct compositions. If administered separately, one may be administered prior to, concurrent with or following administration of the other, or in any combination thereof. Furthermore, the system Xc- inhibitor and statin(s) may be formulated in a controlled release system in which the release of the agents is controlled spatially, temporally or a combination thereof (e.g. the composition may be formulated so that one agent is the first active agent to be released, while the other agent is released sometime thereafter).

A system Xc- inhibitor may also be administered to an individual who has been previously treated with statin therapy to treat statin-induced myalgia, or to an individual who is statin naive but prescribed for statin therapy.

Inhibitors of system Xc- may be provided in a composition comprising one or more additional active ingredients, such as a statin, one or more additional system Xc- inhibitors, a compound effective to treat pain, a compound effective to treat mitochondrial dysfunction, and the like.

A system Xc- inhibitor may be administered in conjunction with at least one other system Xc- inhibitor in accordance with a further embodiment of the invention. As an example, sulfasalazine may be administered in combination with, or simultaneously with vitamin E and/or cysteamine. Other examples of combinations are illustrated herein, but are not limiting.

A system Xc- inhibitor may also be administered in conjunction with at least one compound effective to treat muscle pain. For example, a system Xc- inhibitor may be administered in combination or simultaneously with treatments such as non-steroidal anti-inflammatory agents (e.g. ibuprofen, naproxen sodium, celecoxib and ketoprofen), acetaminophen, tricyclic anti-depressants (e.g. amitryptiline and nortryptiline), anti-convulsants (e.g. gabapentin, pregabalin, valproic acid and topiramate), selective serotonin reuptake inhibitors (e.g. fluoxetine and duloxetine), a muscle heating source, a muscle cooling source, therapeutic massage, cannabinoids, (e.g. cannabidiol) and the like.

A system Xc- inhibitor may also be administered in conjunction with at least one compound effective to treat mitochondrial dysfunction. For example, a system Xc- inhibitor may be administered in combination or simultaneously with treatments such as antioxidants (e.g., EUK-134 and MnTBAP), mitochondrially targeted antioxidants (e.g. MITO Tempo, EPI-743 and elamepratide), thiamine, riboflavin and Coenzyme Q10. Since Coenzyme Q10 functions as both a system Xc- inhibitor and a treatment for mitochondrial dysfunction, it may be desirable to administer an increased dosage thereof to achieve the desired efficacy.

In another aspect of the invention, a kit is provided comprising a pharmaceutical composition for inhibiting system Xc- activity or an individual system Xc- inhibitor in a mammal in combination with one or more additional pharmaceutical compositions comprising one or more statins, one or more compounds effective to treat mitochondrial dysfunction, and one or more compounds effective to treat muscle pain.

In a further embodiment, a method is provided for treating fibromyalgia in a mammal, comprising the administration of a system Xc- inhibitor to the mammal. Fibromyalgia is a common disorder characterized by chronic musculoskeletal pain and is often associated with sleep abnormalities, fatigue and mood impairment. The system Xc- inhibitor may be administered alone, in combination with other system Xc- inhibitors, with one or more pharmaceutical carriers to achieve a particular administrable dosage form, in combination with one or more additional active ingredients (as described above), or any combination thereof. Suitable dosages are above-described with respect to treatment of myalgia.

Embodiments of the invention are described in the following examples which are not to be construed as limiting.

### Example 1—Glutamate Efflux is Increased by Statin Exposure and Reduced b Inhibition of System Xc-

To determine if statin therapy results in an increase in glutamate efflux from skeletal muscle cells and if this is associated with system Xc-, C2C12 myotubes, cultured human myoblasts and rats were treated with a commonly prescribed statin alone or in combination with inhibitors of system Xc-.

C2C12 murine myoblasts (American Type Culture Collection) were seeded in 100-mm culture dishes and maintained at sub-confluent levels in high-glucose (4.5 g/L) Dulbecco modified Eagle medium (DMEM; GIBCO) containing 10% fetal bovine serum (GIBCO) and L-glutamine at 37° C. in a humidified atmosphere of 5% CO₂. C2C12 cells were seeded on 60-mm culture dishes prior to differentiation. Differentiation was induced by replacing the culture medium with high-glucose DMEM containing 2% horse serum (GIBCO) and L-glutamine, daily. Following 5 days of differentiation, atorvastatin calcium (Cayman Chemical) dissolved in 40% DMSO/60% saline solution was added to dishes in a final concentration of 5 μM (an equal volume of 40% DMSO/60% saline was added to control treatments). Sulfasalazine (Sigma-Aldrich) dissolved in 1M NH₄OH was added to dishes at a final concentration of 20 μM. Cysteamine bitartrate, vitamin E, ubiquinol and N-acetylcysteine were dissolved in DMS Hybri-Max (Sigma-Aldrich) and separately added to dishes 48 hr prior to statin treatment. Cysteamine bitartrate, vitamin E, coenzyme Q10 and N-acetylcysteine were added to dishes at a final concentration of 100 μM and 300 μM, 100 μM, 50 μM and 5 mM, respectively. A Vitamin E and coenzyme Q10 combination therapy was added to dishes to achieve a final concentration of 100 μM vitamin E and 35 μM coenzyme Q10. C2C12 cells were harvested by first rinsing twice with cold PBS, then scraping and vigorously triturating in NP-40 lysis buffer supplemented with protease inhibitors (Sigma-Aldrich), sodium orthavanadate and dithiothreitol.

Western blotting was performed as follows. Cell lysates were prepared by 1:1 addition of sample buffer (0.5 M Tris base, 13% glycerol, 0.5% SDS, 13% β-mercaptoethanol, and bromophenol blue). Samples were separated by molecular weight on a 12% acrylamide separating gel overlaid by a 4% acrylamide stacking gel. Separated proteins were then transferred to PVDF membranes and blocked in 5% BSA in Tris-buffered saline and Tween-20 (TBST). Membranes were incubated with polyclonal xCT antibodies (1:1,000 in 5% BSA; Novus Biologicals) and monoclonal Vinculin antibodies (1:1,000 in 5% BSA; Santa Cruz Biotechnology) separately overnight at 4° C. Following overnight incubation, membranes were washed 3 times with TBST for 10 mins per wash and incubated with their respective horseradish peroxidase conjugated secondary antibodies (1:10,000 in 5% BSA) for 1 hour at ambient temperature. Antibodies were detected by enhanced chemiluminescence (Thermo Fisher Scientific). Bands were quantified via densitometry and normalized to vinculin.

For the measurement of glutamate efflux, cell culture media was harvested immediately prior to lysing of cells. Glutamate efflux from myotubes was then determined using the Amplex Red glutamic acid assay kit (Life Technologies) according to manufacturer instructions. Briefly, culture media and Amplex red reagent were added 1:1 to 96-well plates and incubated at 37° C. for 30 min. Following incubation, fluorescence was measured by fluorescence microplate reader (BioTek Synergy HT) using excitation wavelength of 530 nm and emission detection at 590 nm. Absorbance values were corrected for background fluorescence and converted to glutamate concentrations. Pierce BCA protein assay kit (Thermo Fisher Scientific) was used to determine protein concentration of cell lysates by methods described therein. Final glutamate concentrations were normalized to cell lysate protein content.

Cultured human myoblasts were collected at McMaster University with approval of the Hamilton Integrated Research Ethics Board (HIREB) under application #11-114. Human myoblasts were derived from fresh muscle collected from human biopsies. Upon collection, muscle was briefly stored in phosphate-buffered saline (PBS) supplemented with 100 mM D-glucose and placed on ice. Muscle was then transferred to 35-mm culture dishes containing a pre-warmed, freshly prepared digestion solution (1.2 U/ml dispase and 1.5 U/ml collagen IV). After mincing, the muscle was incubated for 45 minutes. Muscle slurry was then washed with glucose-supplemented PBS and spun at 400×g for four minutes. The supernatant was discarded, 10 ml of 0.05% T-EDTA was added, and the solution was incubated for 90 minutes. After incubation, proliferation media was added, and the solution was filtered. Upon initiation of growth, cells were transferred to 35-mm Matrigel-coated wells and left to rest for 72 hours. Differentiation was induced with high-glucose DMEM containing 2% horse serum (GIBCO) and L-glutamine, daily. Following 5 days of differentiation, atorvastatin calcium (Cayman Chemical) dissolved in 40% DMSO/60% saline solution was added to dishes at a final concentration of 5 μM. An equal volume of 40% DMSO/60% saline was added to control treatments. Sulfasalazine (Sigma-Aldrich) dissolved in 1M NH₄OH was added to dishes at a final concentration of 20 μM. Cells were harvested by first rinsing twice with cold PBS, then scraping and vigorously triturating in NP-40 lysis buffer supplemented with protease inhibitors (Sigma-Aldrich), sodium orthavanadate and dithiothreitol.

Cultured human fibroblasts were collected at McMaster University with approval of the Hamilton Integrated Research Ethics Board (HIREB) under application #11-114. Human fibroblasts were derived from skin samples collected from human biopsies of the skin on the inner forearm. Upon collection, the approximately 2 mm skin sample was separated into 9 segments and allowed to dry in a 6-well plate for 5 minutes. Growth media was added and cells were incubated for 4 days. Two ml of media was added to each well, and media was changed every 2 days thereafter until outgrowth of fibroblasts was seen. Once cells became confluent, media was removed and cells were washed with 1×PBS. Trypsin-EDTA (0.05%) was added to separate the cells from their dishes, and cells were placed in a T175 flask at a density of 500 k per flask. Media was changed every 2 days until the flasks became confluent. Differentiation was induced with high-glucose DMEM containing 2% horse serum (GIBCO) and L-glutamine, daily. Following 5 days of differentiation, atorvastatin calcium (Cayman Chemical) dissolved in 40% DMSO/60% saline solution was added to dishes to a final concentration of 5 μM. An equal volume of 40% DMSO/60% saline was added as a control treatment. Cells were harvested by first rinsing twice with cold PBS, then scraping and vigorously triturating in NP-40 lysis buffer supplemented with protease inhibitors (Sigma-Aldrich), sodium orthavanadate and dithiothreitol.

In order to evaluate an association between statins and glutamate efflux in an in vivo model, male CD (Sprague Dawley) IGS Rats (Charles River Laboratories) were provided chow and water ad libitum. Animal housing conditions were maintained at 21° C., 50% humidity, and a 12-h/12-h light-dark cycle. Experimentation was approved by the McMaster University Animal Research Ethics Board, in accordance with the guidelines of the Canadian Council for Animal Care. Rats were randomly assigned into treatment groups and fed 4 g of Nutella (Fererro S.p.A.) containing their respective treatment for a period of 10 days. Control rats received only Nutella in addition to their normal chow diets. Rats in the “Statin” group were administered 40 mg/kg/day of atorvastatin in their Nutella. Rats in the “Statin+SSZ” group, were administered 40 mg/kg/day of atorvastatin with 200 mg/kg/day of sulfasalazine. Rats iii the “Statin+Composition A” and “Statin+Composition B” groups were each administered compositions intended to inhibit system Xc- in a dosage that is based on a fixed percentage of a typical chow diet for a rat. Based on the weights of the rats used in the study, the average rat would be expected to eat 22 g of standard chow per day. Therefore, each of the components in the Composition A and Composition B inhibitors were administered based on a 22 g daily food consumption. Rats in the “Statin+Composition A” group were administered 40 mg/kg/day of atorvastatin with a composition comprising vitamin E (1000 IU/kg of food in addition to the amount in standard chow), coenzyme Q10 (1.25% of diet), beet root extract (1% of diet), alpha lipoic acid (0.1% of diet) and creatine (1% of diet). Rats in the “Statin+Composition B” group were administered 40 mg/kg/day of atorvastatin with a composition comprising vitamin E (1000 lU/kg of food in addition to the amount in standard chow), coenzyme Q10 (1.25% of diet), beet root extract (1% of diet), alpha lipoic acid (0.1% of diet), creatine (1% of diet), green tea extract (0.25% of diet), black tea extract (0.125% of diet), green coffee bean extract (0.25% of diet), conjugated linoleic acid (0.25% of diet) and forskolin (0.005% of diet).

Interstitial dialysis (for dialysate collection and subsequent glutamate analysis) and tissue collection took place on day 10 of treatment. It is important to note that, unlike in the dialysate, systemic elevations in glutamate (i.e., in the blood) will not represent the glutamate pool responsible for nociceptor activation. The microdialysis technique is based on the principle that diffusion occurs across a semi-permeable membrane between the solution that passes through the microdialysis probe (perfusate) and the extracellular fluid surrounding the probe. Subsequently, compounds in the interstitial space can diffuse into the microdialysis probe. Microdialysis probes were constructed on-site to specifications required for this application. The dialysis fiber length was 10 mm, and allowed free diffusion of substances up to 13,000 Daltons. Briefly, hair was removed from the hindlimbs of all animals, and animals were anesthetized via gaseous isoflurane.

While anesthetized, two microdialysis probes were inserted into the gastroenemius muscle of each leg, running in parallel with the long axis of the muscle fibers. To insert the probes, an 18-gauge steel guide cannula was first inserted in a direction parallel to muscle fiber orientation. The dialysis tubing was then fed through the cannula, and the cannula was removed leaving the dialysis tubing in direct contact with the interstitium of the skeletal muscle. The microdialysis probes were perfused (via a perfusion pump; CMA Model 201) at 2 ul/minute with a saline solution. Following a 60-minute equilibration period, dialysate was collected (in three 30-minute blocks) into polyethylene tubes. Following collection, samples were stored at −80° C., and glutamate analysis was conducted, as mentioned above.

### System Xc- Expression is Upregulated in C2C12 Myotubes in Response to Atorvastatin Treatment.

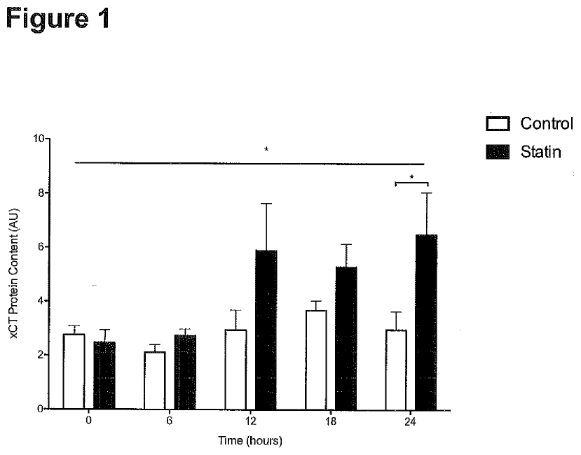

To determine if system Xc- content is altered by statins, expression of the system Xc- subunit xCT was measured following treatment with atorvastatin. C2C12 myotubes were treated with either atorvastatin or vehicle for 0, 6, 12, 18, and 24 hours. The cells were then lysed, and expression of xCT was quantified by chemiluminescent immunoblot. After only 12 hours, myotubes treated with atorvastatin displayed an approximately 2-fold elevation in xCT abundance (FIG. 1). This elevated xCT protein level was still present in the atorvastatin group at the 24 hour treatment time point. As expected, xCT protein content in vehicle-treated cells remained at the baseline level throughout the treatment period. These data demonstrate that statins cause an increase in system Xc- content.

### Glutamate Release in Muscle Cells In Vivo and In Vitro is Increased in Response to Atorvastatin Treatment and Sensitive to Inhibition of System Xc-

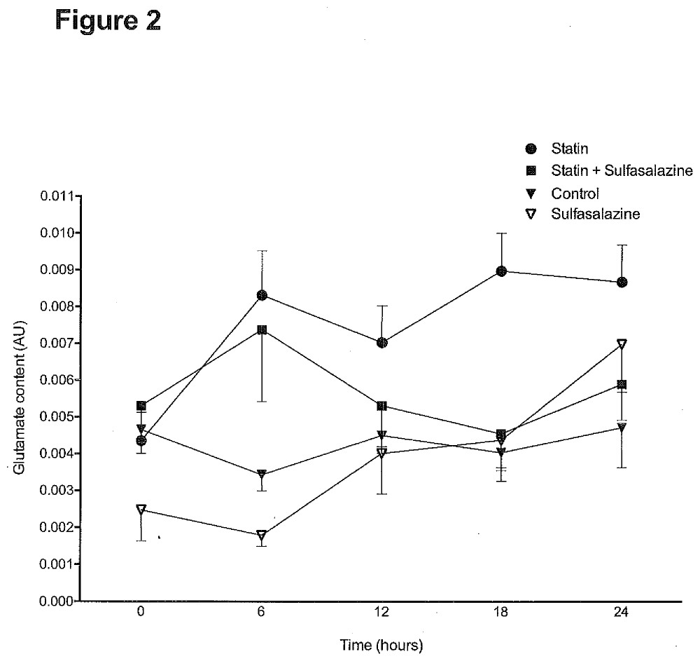

To determine if glutamate efflux is altered by statin treatment and if this effect could be reversed by inhibition of system Xc-, C2C12 myotubes were treated separately with atorvastatin, sulfasalazine (an inhibitor of system Xc- activity), vehicle and an atorvastatin/sulfasalazine co-treatment for 0, 6, 12, 18, and 24 hours. Cell culture media was collected post-treatment immediately prior to harvesting of cells. Myotubes treated with atorvastatin displayed a substantial increase in glutamate efflux at 6 h, an effect which was maintained at the 12 h, 18 h and 24 h time points (FIG. 2). An initial increase of glutamate efflux levels was also seen in the group co-treated with atorvastatin and sulfasalazine at the 6 hour time point. Interestingly, the glutamate efflux levels declined to baseline levels in this co-treated group by the 12 hour time point and remained at baseline levels until the last time point of 24 hour. Both the sulfasalazine alone group and the vehicle control group demonstrated glutamate efflux around those of baseline values throughout the treatment period.

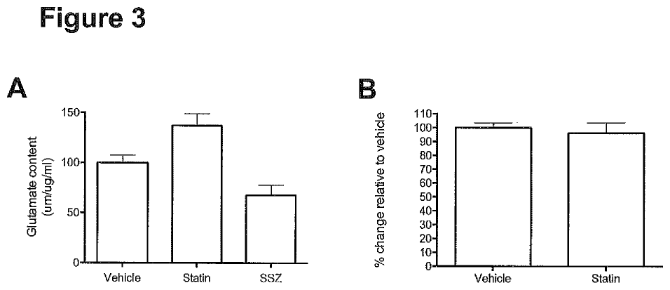

In order to evaluate if the statin-induced increase in glutamate efflux occurs similarly in human cell lines, we next treated primary human myoblasts and fibroblasts (derived from human muscle and skin samples respectively) with statins. Consistent with the observations of FIG. 2, statin administration increased glutamate efflux from human myoblasts, while the co-treatment of statins with the system Xc- inhibitor sulfasalazine blocked this efflux and further reduced extracellular glutamate levels to those below the vehicle control group (FIG. 3A). Surprisingly, no changes in extracellular glutamate concentrations occurred when statins were administered to primary human fibroblasts (FIG. 3B). These findings indicate that statin-induced increase in glutamate efflux does occur in human cell lines, but that the cellular events are limited to skeletal muscle, consistant with localization of pain in individuals experiencing statin-induced myalgia.

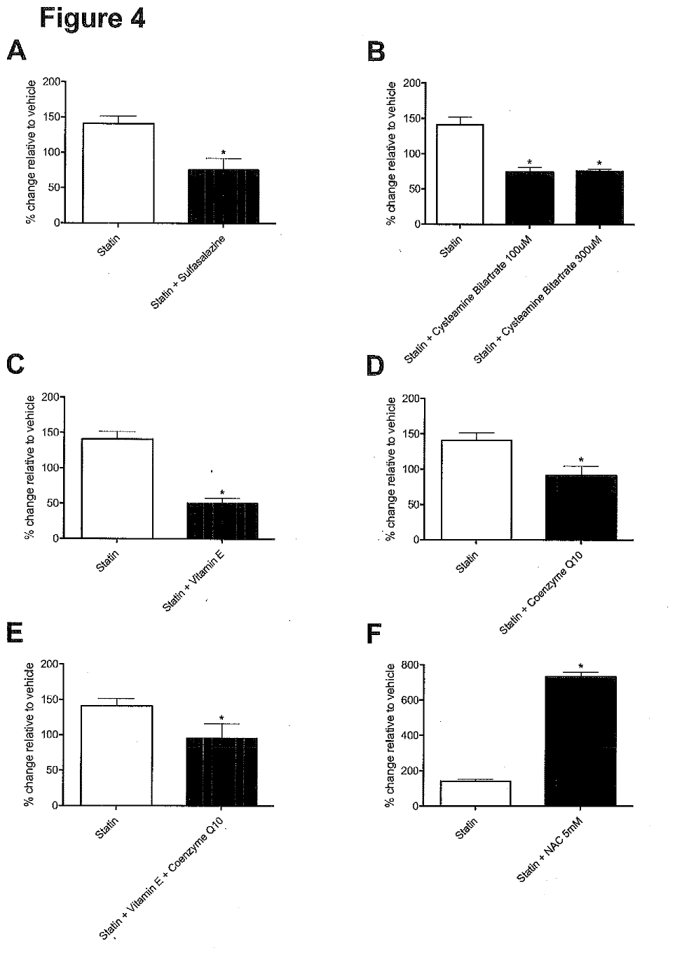

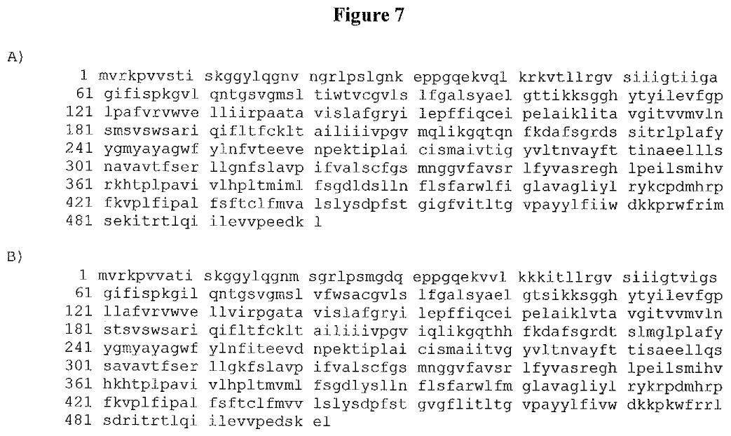

To determine if other compounds (sytem Xc- inhibitors) could be used to reduce glutamate efflux from muscle cells, several other agents were administered to atorvastatin-treated C2C12 myotubes. Reconfirming the findings from FIGS. 2 and 3, sulfasalazine inhibited glutamate efflux caused by both 5 μM (FIG. 4A) and 7.5 μM (FIG. 5A) atorvastatin treatment. Cysteamine bitartrate, vitamin E, ubiquinol and a combination of vitamin E with ubiquinol all mitigated the increase in glutamate efflux caused by 5 μM (FIG. 4 B-E respectively) and 7.5 μM (FIG. 5 B-E respectively) atorvastatin treatment. Conversely, the antioxidant N-acetylcysteine (NAC) substantially increased the glutamate efflux caused by 5 μM (FIG. 4F) and 7.5 μM (FIG. 5F) atorvastatin.

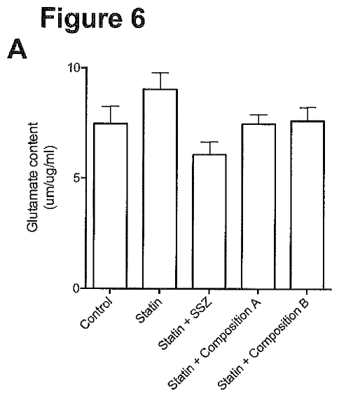

In order to confirm that these results were translatable to an in vivo model, statins and various system Xc- inhibitors were orally administered to Sprague Dawley rats for a duration of 10 days. Glutamate efflux was measured via the interstitial dialysis technique in the lower leg muscles as described above. All rats were assigned to one of the following experimental groups: no treatment (group referred to as “Control”), statins (group referred to as “statins”), statins with sulfasalazine (group referred to as “Statin+SSZ”), statins with a system Xc- inhibitor composition comprising vitamin E, coenzyme Q10, beet root extract, alpha lipoic acid and creatine (group referred to as “Statin+Composition A”) and statins with a system Xc- inhibitor composition comprising vitamin E, coenzyme Q10, beet root extract, alpha lipoic acid, creatine, green tea extract, black tea extract, green coffee bean extract, conjugated linoleic acid and forskolin (group referred to as “Statin+Composition B”). Compared with the Control group, rats in the Statin group experienced a 21% increase in extramyocellular glutamate concentrations (FIG. 6). As observed in vitro, sulfasalazine administration with statins reduced extramyocellular glutamate concentrations substantially (19% lower than Control). The rise in glutamate efflux from statins was also almost completely prevented (less than 2% change from Control) in rats administered the Statin+Composition A and Statin+Composition B.

These findings confirm that the inhibition of system Xc- is an effective strategy for reducing excessive glutamate efflux from muscle that is caused by statin therapy.

1. A method of reducing glutamate efflux from muscle cells comprising the step of administering a system Xc- inhibitor to the cells.

2. (canceled)

3. The method of claim 1, wherein the system Xc- inhibitor is selected from the group consisting of: sulfasalazine, vitamin E, coenzyme Q10, cysteamine and combinations thereof.

4. (canceled)

5. The method of claim 1, wherein glutamate efflux is reduced by at least about 25% of the glutamate efflux caused by statin administration.

6. The method of claim 3, wherein sulfasalazine is administered at a dosage in the range of about 250 mg to about 5,000 mg; Vitamin E is administered at a dosage in the range of about 25 IU to about 2,500 IU; cysteamine is administered at a dosage in the range of about 150 mg to about 6,000 mg; and coenzyme Q10 is administered at a dosage in the range of about 25 mg to about 1,000 mg.

7. (canceled)

8. The method of claim 1, wherein the system Xc- inhibitor is administered in conjunction with a statin.

9. (canceled)

10. The method of claim 1, wherein the second therapeutic agent is effective to treat muscle pain and is selected from the group of: non-steroidal anti-inflammatory agents, acetaminophen, tricyclic anti-depressants, anti-convulsants, selective serotonin reuptake inhibitors, and cannabinoids.

11. The method of claim 1, wherein the second therapeutic agent is effective to treat mitochondrial dysfunction and is selected from the group of: antioxidants, mitochondrially targeted antioxidants, thiamine and riboflavin.

12. A method of treating statin-induced myalgia in a mammal comprising the step of administering to the mammal a system Xc- inhibitor.

13. (canceled)

14. The method of claim 12, wherein the system Xc- inhibitor is selected from the group consisting of: sulfasalazine, vitamin E, coenzyme Q10, cysteamine and combinations thereof.

15. (canceled)

16. The method of claim 12, wherein the system Xc- inhibitor is administered in conjunction with a statin.

17. The method of claim 16, wherein the system Xc- inhibitor is administered in conjunction with a second therapeutic agent.

18. The method of claim 16, wherein the second therapeutic agent is effective to treat muscle pain and is selected from the group of: non-steroidal anti-inflammatory agents, acetaminophen, tricyclic anti-depressants, anti-convulsants, selective serotonin reuptake inhibitors, a muscle heating source, a muscle cooling source and cannabinoids, or the second therapeutic agent is effective to treat mitochondrial dysfunction and is selected from the group of: antioxidants, mitochondrially targeted antioxidants, thiamine and riboflavin.

19. (canceled)

20. A composition useful for treating statin-induced myalgia in a mammal comprising a system Xc- inhibitor in combination with i) one or more additional Xc-inhibitors; ii) a statin; or iii) a second therapeutic agent.

21. (canceled)

22. The composition of claim 20, wherein the system Xc- inhibitors are selected from the group consisting of: sulfasalazine, vitamin E, coenzyme Q10, cysteamine and combinations thereof.

23. The composition as defined in claim 20, comprising a statin.

24. The composition as defined in claim 20, comprising a second therapeutic agent.

25. The composition of claim 24, wherein the second therapeutic agent is effective to treat muscle pain and is selected from the group of: non-steroidal anti-inflammatory agents, acetaminophen, tricyclic anti-depressants, anti-convulsants, selective serotonin reuptake inhibitors, and a cannabinoid, or the second therapeutic agent is effective to treat mitochondrial dysfunction and is selected from the group of: antioxidants, mitochondrially targeted antioxidants, thiamine and riboflavin.

26. (canceled)

27. A kit useful to treat statin-induced myalgia comprising a system Xc-inhibitor and a statin.

28. (canceled)

29. The method of claim 12, wherein the system Xc- inhibitor comprises a combination of vitamin E, coenzyme Q10, beet root extract, alpha lipoic acid and creatine.

30. The composition of claim 20, comprising a combination of vitamin E, coenzyme Q10, beet root extract, alpha lipoic acid and creatine.
